(19) United States
(12) Patent Application Publication
(10) Pub. No.: US 2021/0220491 A1
(43) Pub. Date: Jul. 22, 2021

(54) FLUORESCENCE IMAGING METHOD AND SYSTEM

(71) Applicants: Shyh-Chuan Jwo, Keelung City (TW); Chung Yuan Christian University, Taoyuan City (TW); National Central University, Taoyuan City (TW)

(72) Inventors: Ming-Fa Hsieh, Hsinchu County (TW); Shang-Ting Tsai, New Taipei City (TW); Tzu-Chau Lin, Taoyuan City (TW); Wen-Tyng Li, Taoyuan City (TW); Tzong-Rong Ger, Taoyuan City (TW); Shyh-Chuan Jwo, Keelung City (TW)

(21) Appl. No.: 17/150,488

(22) Filed: Jan. 15, 2021

(30) Foreign Application Priority Data
Jan. 21, 2020 (TW) 109102228

(51) Int. Cl. A61K 49/00

(52) U.S. Cl. CPC A61K 49/0054; A61K 49/0021

(57) ABSTRACT

This invention provides an imaging method for detecting a volume of animal or human to obtain a fluorescence image of the volume. The method comprises steps of: treating the animal or human with an dosage form containing a dye encapsulated by a polymer; irradiating the volume of the animal or human by a light, and detecting a single photon, two- or multi-photon fluorescence emission light from the illuminated volume to obtain a fluorescence image, wherein a peak wavelength of the single photon fluorescence emission light of the dosage form is equal to or greater than 780 nm, or a peak wavelength of the two or multi-photon fluorescence emission light of the dosage form is equal to or greater than 800 nm. This invention further provides an imaging system employing the foregoing imaging method.

### CROSS-REFERENCE TO RELATED APPLICATIONS

The entire contents of Taiwan Patent Application No. 109102228, filed on Jan. 21, 2020, from which this application claims priority, are expressly incorporated herein by reference.

### BACKGROUND OF THE INVENTION

### 1. Field of the Invention

The present invention relates to a fluorescence imaging method and a fluorescence imaging system, especially to a fluorescence imaging method and a fluorescence imaging system for detecting a fluorescence image of an animal or a human body with a dosage form.

### 2. Description of Related Art

Several conventional methods, such as ultrasound imaging and magnetic resonance imaging, are employed for tumor diagnosis and evaluation of surgical resection margin. Because the imaging procedure and instruments are complex and expensive, they are difficult to realize real-time monitoring and evaluating during surgery.

Recently, fluorescent imaging methods have been gradually taken seriously in the past decade. Taking clinically peritumoral lymph node metastasis as an example, the status of the axillary and/or sentinel lymph nodes is one of the strongest prognostic factors in women with early-stage breast cancer, and the sentinel lymph node biopsy (SLNB) has become the standard evaluation process of tumor metastasizing to the lymph node basin. The sentinel lymph node is known as the first node in the lymphatic basin that receives drainage from an anatomic region and is immunologically responsible for that region. Sentinel lymph node is very close to the primary lesion and is the first region the metastasizing cancer cells spread to. The SLNB is a surgical procedure for evaluating the spread of a primary tumor of breast cancer into the lymph node, in which the lymph node mapping plays an important role. Commercially, hydrophilic fluorescent molecular probes such as green fluorescence dyes and radio-colloids have been used for metastatic lymph node mapping and lymphatic transport in lymphedema, tumor localization and surgical margin evaluation. However, these probes have several limitations such as moderate to poor photostability (photo-bleaching), undesired reactivity with nucleophiles, propensity to self-quenching, and hazardous radiation exposure. In addition, blue dyes have been used for lymphatic node mapping, but still have associated disadvantages such as difficult detection in pigmented nodes, rapid migration beyond sentinel lymph node and anaphylaxis reaction. On the other hand, the radiocolloid with larger particle size had to be injected 24 hrs before the surgery and the patients and doctors may be exposed to the radiation hazard.

In addition, the commercially available dyes also have low two-photon cross sections (δ, expressed in Goeppert-Mayer (GM)=1×⁻⁵⁰ photon cm⁴ s photon⁻¹ molecule⁻) which is less than 200 GM.

### SUMMARY OF THE INVENTION

This invention relates to a fluorescence imaging method or system to detect a volume of an animal or a human body and then obtain a fluorescence image of the volume. The fluorescence imaging method or system comprises: treating an animal or a human body with a dosage form including a dye encapsulated with a polymer; irradiating a volume of the animal or the human body by an excitation light, the dosage form absorbing two or multi-photons of the excitation light and re-emitting a fluorescence emission light with a peak wavelength equal to or greater than 800 nm, and detecting the fluorescence emission light from the volume to obtain a fluorescence image.

In some embodiments, the polymer is a copolymer derived from more than one species of monomer. In some embodiments, the polymer is an amphiphilic copolymer including a hydrophilic group and a lipophilic group. In some embodiments, both ends of the copolymer are hydrophilic groups.

In a preferred embodiment, the fluorescence imaging method further comprises: irradiating the volume of the animal or the human body by a visible light and detecting light from the volume to obtain a bright field image; and merging the fluorescence image and the bright field image to obtain a merged image.

In some embodiments, a maximum two-photon cross section of the dye in a wavelength range from 700 nm to 1000 nm is larger than 1800 GM.

In some embodiments, the peak wavelength of the excitation light is adjusted so that the dosage form absorbs single-photons of the excitation light and re-emits a fluorescence emission light with a peak wavelength equal to or greater than 780 nm.

### BRIEF DESCRIPTION OF THE DRAWINGS

FIG. 1 is a schematic view showing a fluorescent imaging system in accordance with an embodiment of the present invention.

FIGS. 2A and 2B are flow charts showing a fluorescent imaging method in accordance with an embodiment of the present invention.

FIG. 3A shows a formula of dye in accordance with an embodiment of the present invention.

FIG. 3B shows a formula of dye in accordance with an embodiment of the present invention.

FIG. 4 shows a formula of dye in accordance with an embodiment of the present invention.

FIG. 5 shows a formula of dye in accordance with an embodiment of the present invention.

FIG. 6 shows a formula of dye in accordance with an embodiment of the present invention.

FIG. 7 shows a formula of dye in accordance with an embodiment of the present invention.

FIG. 8 shows a formula of dye in accordance with an embodiment of the present invention.

FIG. 9 shows the synthesis of the dye in FIG. 7.

FIG. 10A shows the single-photon linear optical properties of the dye in FIG. 7.

FIG. 10B shows the two-photon linear optical properties of the dye in FIG. 5.

FIG. 10C shows the single-photon linear optical properties of the dye in FIG. 8.

FIG. 11 shows the two-photon absorption cross-section of the dye of FIG. 7 measured with a femtosecond laser.

FIG. 12 shows the structure formula of an amphiphilic copolymer in accordance with an embodiment of the present invention.

FIG. 13 is a schematic diagram showing a dye encapsulated in an amphiphilic copolymer to form a dosage form in accordance with an embodiment of the present invention.

FIG. 14 shows that the dye of FIG. 6 absorbs human breast cancer cells and emits near-infrared fluorescence light (displayed in red through image processing).

FIG. 15 is a cell viability assay (MTT assay) of a dosage form in accordance with an embodiment of the present invention.

FIG. 16 is an in vivo imaging of a mouse injected with a dosage form in accordance with an embodiment of the present invention.

### DETAILED DESCRIPTION OF THE PREFERRED EMBODIMENT

Reference will now be made in detail to those specific embodiments of the invention. Examples of these embodiments are illustrated in accompanying drawings. While the invention will be described in conjunction with these specific embodiments, it will be understood that it is not intended to limit the invention to these embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well-known process operations and components are not described in detail in order not to unnecessarily obscure the present invention.

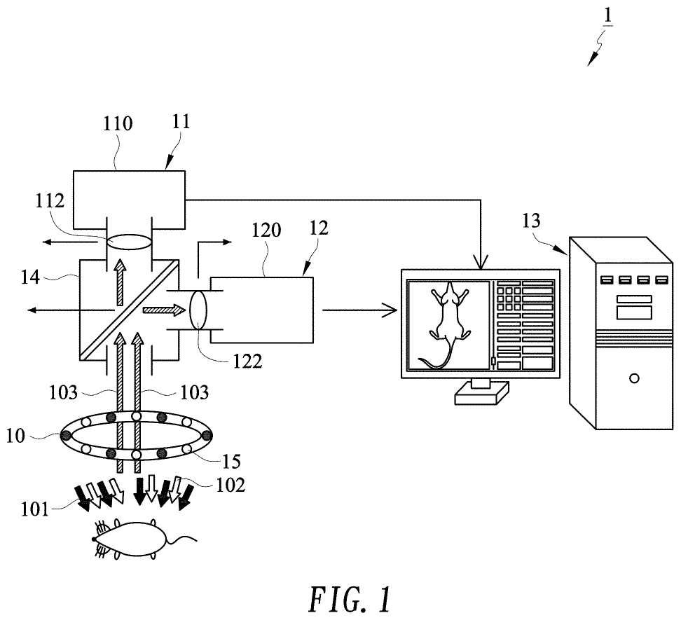

FIG. 1 is a schematic diagram showing a fluorescent imaging system 1 in accordance with an embodiment of the present invention. As shown in FIG. 1, the fluorescent imaging system 1 mainly includes a first light source 10, a fluorescent image capturing unit 11, and an image processing system 13. A dosage form (not shown) is injected into a human body or animal and reaches a volume of the human body or animal. In this context, “volume” refers to a part or all of an organ or tissue of the human body or animal in vivo or in vitro.

An excitation light 101 of the first light source 10 illuminates the volume. In one embodiment, the excitation light is a continuous near infrared light. In another embodiment, the excitation light is a pulsed near infrared light. The dosage form absorbs the excitation light 101 and then re-emits an emission light 103 to be transmitted to the fluorescent image capturing unit 11.

The fluorescent image capturing unit 11 is used to capture a fluorescent image containing the volume over a first wavelength range. The fluorescent image capturing unit 11 may include a first image sensor 110 and a first filter 112. The first image sensor 110 may be a charge coupled device (CCD) or a complementary metal-oxide semiconductor (CMOS). The first filter 112 selectively transmits light in the first wavelength range, while absorbs the remainder. The first wavelength range may be from about 700 nm to about 1000 nm. In some embodiments, the first filter 112 can adjust the particular range of wavelengths (i.e., the first wavelength range) that passes the first filter 112 according to a peak wavelength of the emission light 103. The peak wavelength of the excitation light 101 can be adjusted according to the desired peak wavelength of the emission light 103.

Referring to FIG. 1, in one embodiment, the fluorescent imaging system 1 may further include a second light source 15 and a bright-field image capturing unit 12. The second light source 15 is used to emit a visible light 102 to illuminate the volume. At this time, the emission light 103 includes the fluorescent light emitted by the dosage form, and further includes emitted, reflected, refracted, and scattering lights (and not limited to these) from the volume after being illuminated with the visible light 102. The first light source 10 and the second light source 15 may have a configuration different from that shown in FIG. 1, and they can illuminate either alternatively or simultaneously. In one embodiment, the second light source 15 is an indoor ambient light. The bright-field image capturing unit 12 is used for capturing a bright-field image including the volume over a second wavelength range. The bright field image capturing unit 12 may include a second image sensor 120 and a second filter 122. The second image sensor 120 may be a charge coupled device (CCD) or a complementary metal-oxide semiconductor (CMOS). The second filter 122 can selectively transmits light in the second wavelength range, while absorbs the remainder. In some embodiments, the second wavelength range may be from about 380 nm to about 750 nm.

Referring to FIG. 1, in one embodiment, the fluorescent imaging system 1 may further include a beam splitter 14 for directing part of the emission light 103 to the bright-field image capturing unit 12 and directing part of the emission light 103 to the fluorescent image capturing unit 11.

Referring to FIG. 1, in one embodiment, the captured fluorescent image and bright-field image are transmitted to an image processing system 13, which combines the bright-field image and the fluorescent image into a merged image. Fluorescent images and bright-field images can be continuously captured in real time, and the image processing system 13 can continuously combine the fluorescent images and the bright-field images into merged images and display them in real time.

Referring to FIG. 1, the dosage form comprises a dye encapsulated by a polymer to absorb single photon, two-photon, or multi-photon of the excitation light 101 and then to re-emit the emission light 103. In some embodiments, the dye comprises quinoxaline derivatives, 2,1,3-benzothiadiazole (BTD) derivatives, or a combination of the above derivatives. In some embodiments, the polymer is a copolymer derived from more than one species of monomer. In some embodiments, the polymer is an amphiphilic copolymer including a hydrophilic group and a lipophilic group. In some embodiments, both ends of the copolymer are hydrophilic groups.

In one embodiment, the polymer is a copolymer or an amphiphilic copolymer, and the copolymer or the amphiphilic copolymer includes a functional group to bind to specific tissues, such as cancer cells of the animal or human body.

In one embodiment, the image processing system 13 executes a software to determine the location of a tumor, and mark it on the merged image. Accordingly, medical staff can conveniently perform subsequent medical procedures, such as tumor resection.

In one embodiment, during the treatment of the volume, at least one chemical is injected with the dosage form to treat the volume of the animal or human body. The at least one chemical may include antibiotics, cancer treatment drugs, and the like.

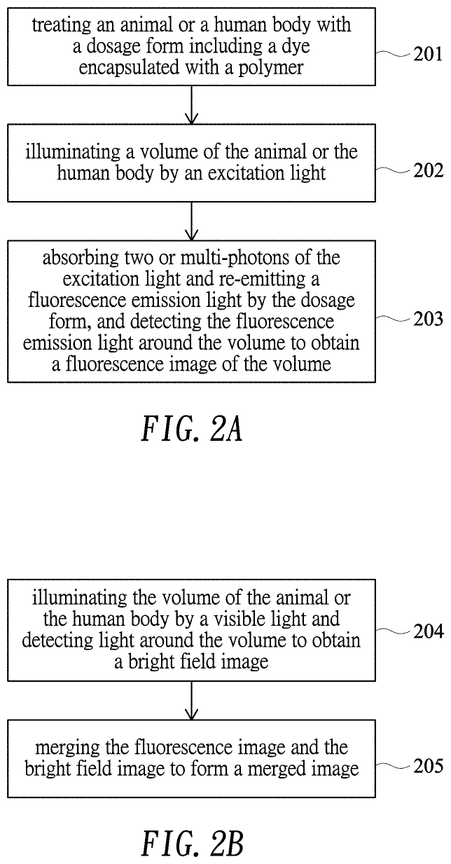

FIG. 2A is a flowchart showing a fluorescent imaging method in accordance with an embodiment of the present invention. As shown in FIG. 2A, the fluorescence imaging method includes: step 201, treating an animal or a human body with a dosage form containing a fluorescent dye encapsulated with a polymer; step 202, irradiating a volume of the animal or the human body by an excitation light; step 203, absorbing two or multi-photons of the excitation light and re-emitting a fluorescence emission light by the dosage form, and detecting the fluorescence emission light from the volume to obtain a fluorescence image of the volume. Preferably, a peak wavelength of the fluorescence emission light is equal to or greater than 800 nm.

Referring to FIG. 2B, in another embodiment, the fluorescent imaging method may further include: step 204, irradiating the volume of the animal or the human body by a visible light and detecting light from the volume to obtain a bright field image; and step 205, merging the fluorescence image and the bright field image to form a merged image.

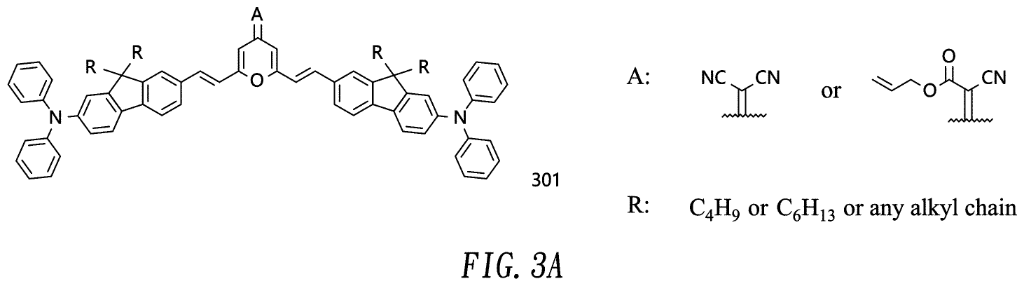

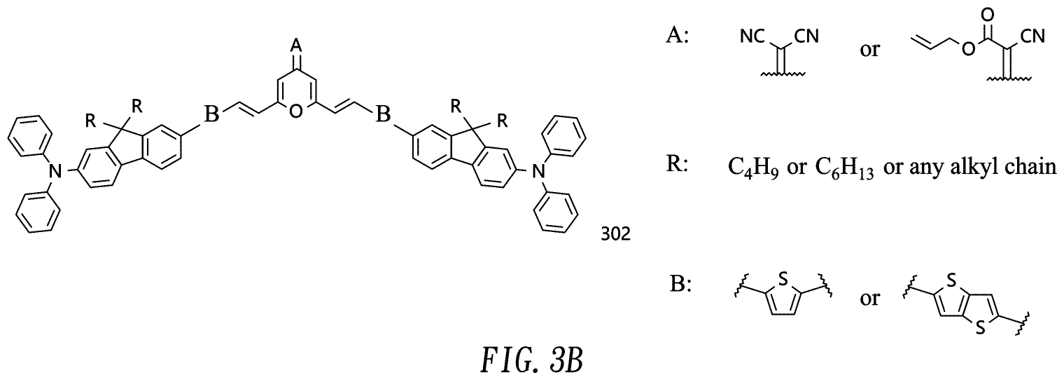

FIG. 3A shows a formula of dye 301 in accordance with an embodiment of the present invention.

FIG. 3B shows a formula of dye 302 in accordance with an embodiment of the present invention.

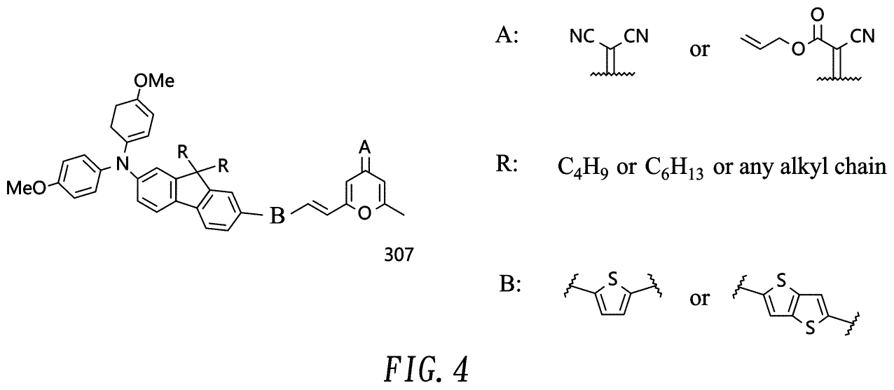

FIG. 4 shows a formula of dye 307 in accordance with an embodiment of the present invention.

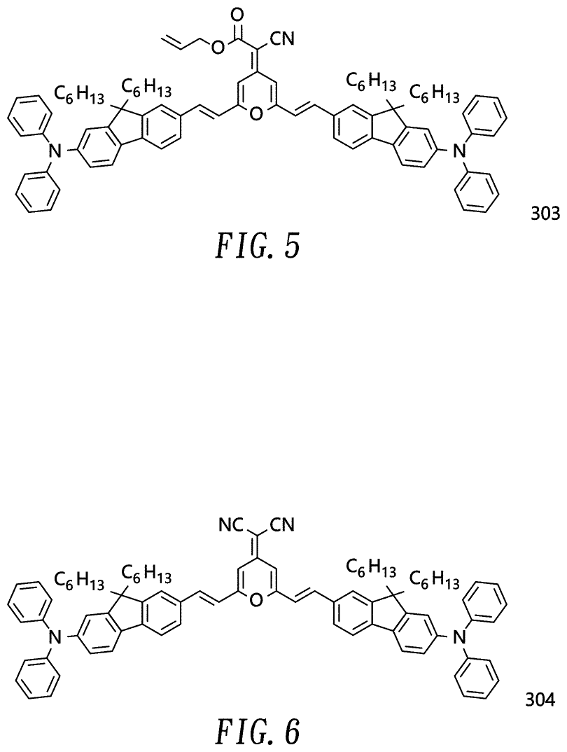

FIG. 5 shows a formula of dye 303 in accordance with an embodiment of the present invention.

FIG. 6 shows a formula of dye 304 in accordance with an embodiment of the present invention.

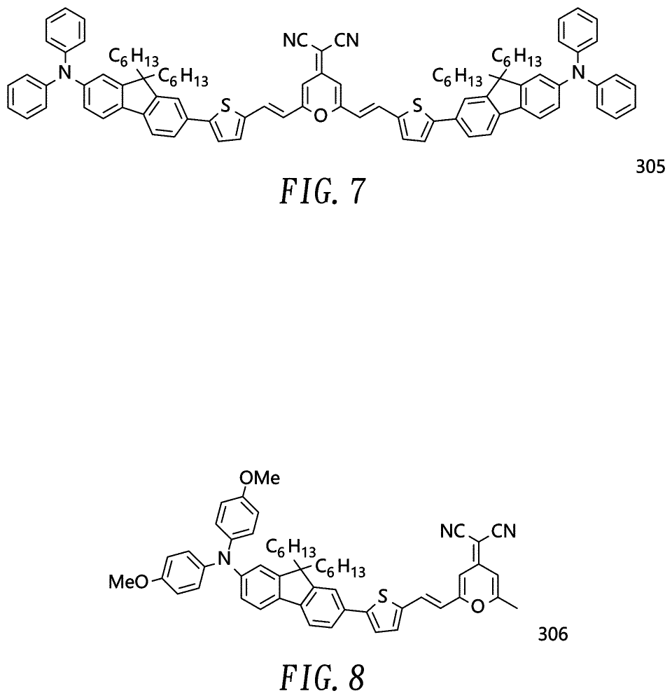

FIG. 7 shows a formula of dye 305 in accordance with an embodiment of the present invention.

FIG. 8 shows a formula of dye 306 in accordance with an embodiment of the present invention.

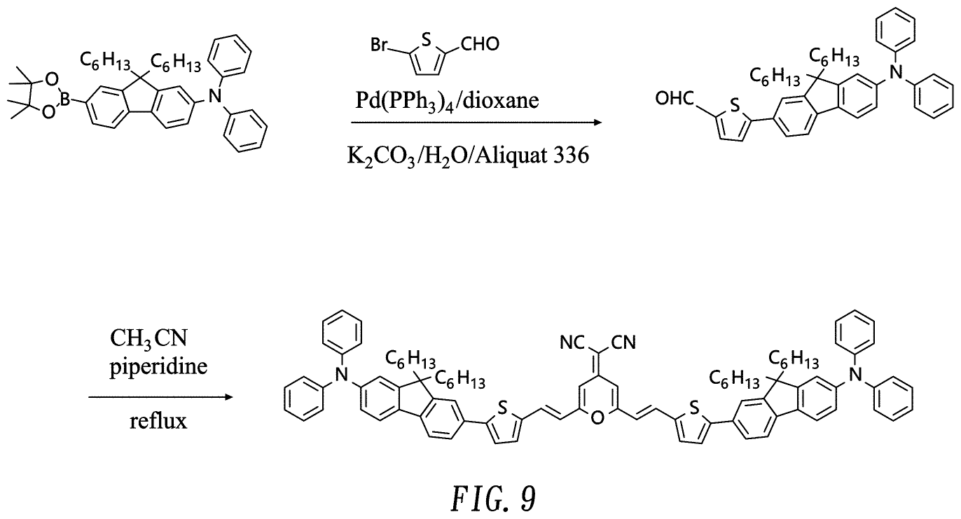

FIG. 9 shows the synthesis of the dye 305 shown in FIG. 7.

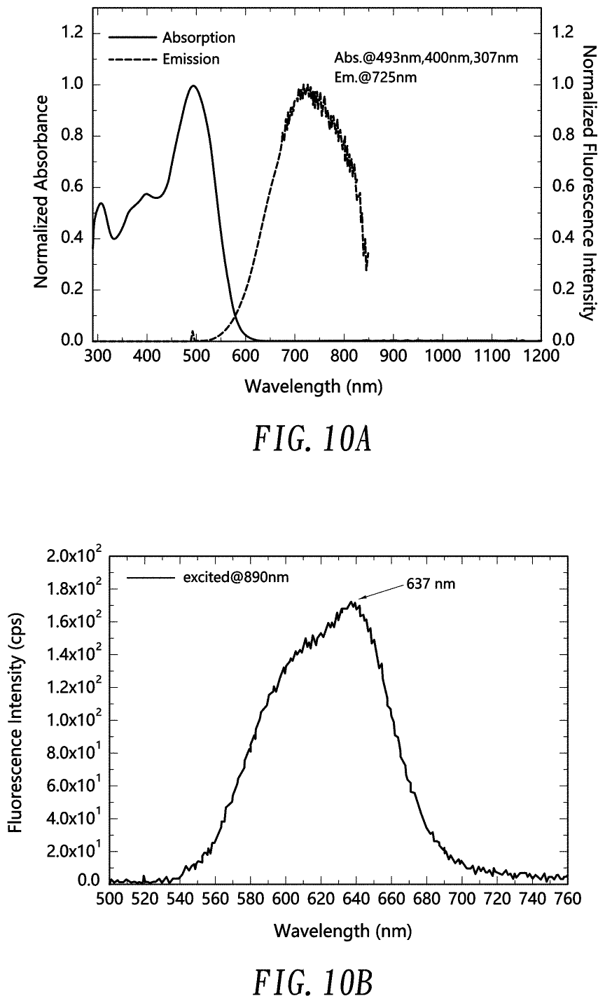

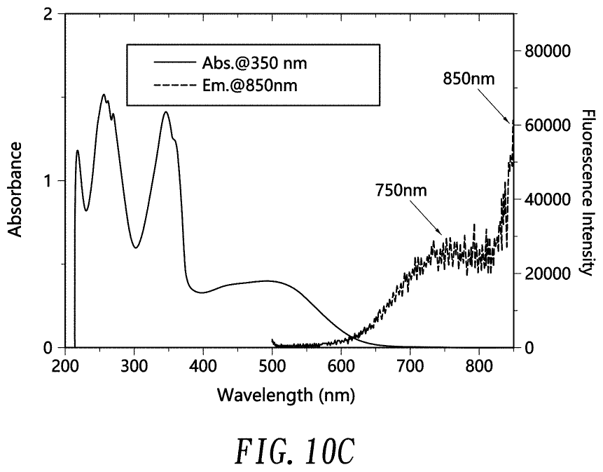

FIG. 10A shows the single-photon linear optical properties of the dye 305 shown in FIG. 7. As shown in FIG. 10A, the excitation light has a major peak with a peak wavelength of 493 nm and two minor peaks with minor peak wavelengths of 400 nm and 307 nm, respectively. The dye absorbs single-photons of the excitation light and then re-emits a fluorescent emission light with a peak wavelength of 725 nm.

FIG. 10B shows the two-photon linear optical properties of the dye 303 shown in FIG. 5. As shown in FIG. 10B, the peak wavelength of the excitation light is 890 nm. The dye absorbs two-photons of the excitation light and then re-emits a fluorescent emission light with a peak wavelength of 637 nm.

FIG. 10C shows the single-photon linear optical properties of the dye 306 shown in FIG. 8. As shown in FIG. 10C, the peak wavelength of the excitation light is 350 nm. The dye absorbs single-photons of the excitation light and then re-emits a fluorescent emission light including a major peak with a peak wavelength of 850 nm and a minor peak with a minor peak wavelength 750 nm.

In one embodiment, the dosage form absorbs two-photons or multi-photons of the excitation light and then re-emits a fluorescent emission light with a peak wavelength equal to or greater than 800 nm. In one embodiment, the peak wavelength of the fluorescent emission light is equal to or greater than 830 nm. In one embodiment, the peak wavelength of the fluorescent emission light is equal to or greater than 850 nm. In one embodiment, the peak wavelength of the fluorescent emission light is equal to or greater than 900 nm.

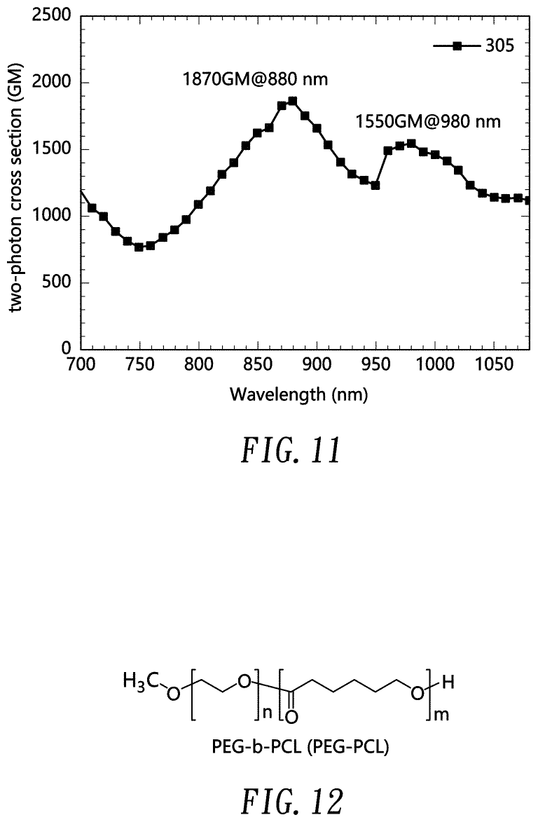

FIG. 11 shows the two-photon absorption cross-section of the dye 305 dissolved in toluene where the cross-section is measured with a femtosecond laser. As shown in FIG. 11, the dye provided by the present invention has a maximum two-photon cross section greater than 1800 GM in the wavelength range from 700 nm to 1000 nm. In some embodiments, the dye provided by the present invention has a maximum two-photon cross section greater than 1900 GM in the wavelength range from 700 nm to 1000 nm. In some embodiments, the dye provided by the present invention has a maximum two-photon cross section greater than 2000 GM in the wavelength range from 700 nm to 1000 nm.

FIG. 12 shows the structure formula of a polymer for encompassing the dye in accordance with an embodiment of the present invention. In this embodiment the polymer is, but is not limited to, an amphiphilic copolymer such as polyethylene glycol-b-poly ε-caprolactone (PEG-b-PCL) block copolymer.

In some embodiments, the polymer used to encompass the dye is derived from one or more the following materials: poly(ethylenimine), poly(aspartic acid), poly(acrylic acid), dextran, cyclodextran, cyclodextrin, polyethylene glycol/(PEG), poly(ethylene oxide)/(PEO), poly(oxyethylene)/(POE), poly(propylene oxide)/(PPO), methoxy poly(ethylene glycol) (mPEG), poly ε-caprolactone (PCL), methoxy poly(ethylene glycol)-poly ε-caprolactone (mPEG-PCL), poly(dithienyl-diketopyrrolopyrrole)/(PDPP), poly (N-vinyl pyrrolidone), (PVP), poly(N-isopropyl acrylamide)/(pNIPAAm), poly(propylene oxide)/(PPO), poly(L-lactide), poly(lactide-co-glycolic acid)/(PLGA), poly(L-aspartic acid)/pAsp, poly(L-histidine), poly(β-amino ester)/(PbAE), disteroyl phosphatidyl ethanolamine/(DSPE), poly(ethylene glycol)-b-poly(lactide)/PELA), poly(ethylene glycol)-b-poly(D,L-lactide)-b-poly(β-amino ester/(PELA-PBAE), N-decanoyl-N-methylglucamine/(MEGA-10), Pluronic®F-127(CAS:9003-11-6), sodium dodecyl sulfate (SDS), sodium dodecylbenzenesulfonate (SDBS), alkyltrimethylammoniun bromides (C₁₀TAB), C₁₂TAB, C₁₄TAB, C₁₆TAB, alkyltriphenylphosphonium bromide (C₁₂TPB, C₁₄TPB, or C₁₆TPB), or a combination thereof.

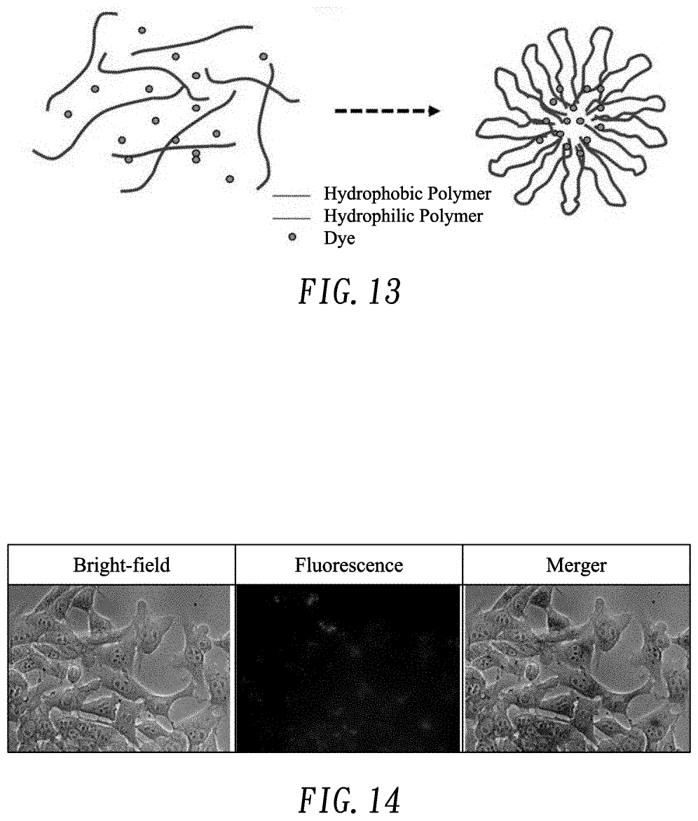

FIG. 13 is a schematic diagram showing a dye encapsulated in an amphiphilic copolymer to form a dosage form in accordance with an embodiment of the present invention. In this embodiment, the amphiphilic copolymer includes a hydrophobic polymer and a hydrophilic polymer.

FIG. 14 shows a bright-field image, a fluorescence image, and a merged image thereof that are captured and merged after treatment of a volume of a human body with a dosage form in accordance with an embodiment of the present invention. As shown in FIG. 14, the dye of the dosage form provided by the embodiment of the present invention is taken up by human breast cancer cells (4T1 cells) and emits near-infrared fluorescence.

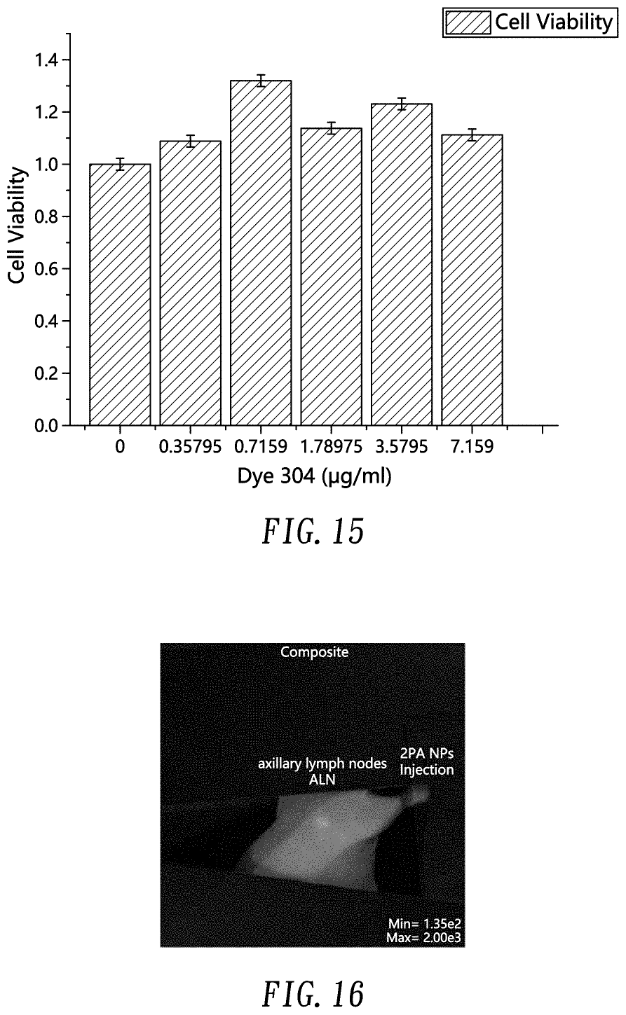

FIG. 15 is a cell viability assay (MTT assay) of a dye shown in FIG. 6. The experiment was performed with human breast cancer cells (4T1 cells) that incubate with dye of FIG. 6. The abscissa refers to the concentration of the dosage form, and the ordinate refers to the cell viability after 24-hr incubation. The test results confirmed that the dye did not reveal signs of acute toxicity in 4T1 cells.

The following demonstrates a specific example:

One BALC/c mouse is anesthetized and then the mouse is shaved and lying down. Next, a microinjector is used to take 10 μL of the invented compound 301 with a concentration of 50 μmol/L. Subcutaneously inject the mouse through the fat pad of a palm, and then gently massage the injection site. Twenty-four hours after the injection, observe the mouse by an in vivo imaging device. The peak wavelength of the excitation light is 460 nm, the peak wavelength of fluorescent emission light is 710 nm, and exposure time for the dye is 20 seconds.

FIG. 16 is an in vivo image of a mouse taken at 24 hours after the subcutaneous injection of the invented dye through the right palm of the mouse. As shown in FIG. 16, obvious fluorescence signals are detected at the lymph nodes near the axilla of the mouse, perceiving a contrast with the surrounding tissues, and the fluorescence signals could still be seen in the lymph nodes after 24 hours. In addition, 1% methylene blue is used as a control to inject the mouse at the same site, and the result confirms that the fluorescence site is the sentinel lymph node draining from the injection site. It confirms that nanoparticles of the invented dosage form can be delivered through the fat pad and the lymphatic vessels, and then reach and fluoresce at the lymph node.

The dosage forms provided by the embodiments of the present invention have the following features:

(1) It has superior optical frequency conversion efficiency. It can absorb two-photons of long-wavelength near-infrared light with longer wavelength than the re-emitted short-wavelength near-infrared light and visible light;

(2) It is highly optical stable and can be exposed to light for a long time without degradation;

(3) It can be effectively encapsulated in polymer micelles without fluorescence quenching due to molecular aggregation.

(4) It can absorb two-photons or multi-photons of the excitation light and then re-emit a fluorescent emission light with a peak wavelength equal to or greater than 800 nm.

The provided fluorescent imaging method and system have various practical applications, such as medical surgery and post-operative check, health examination, medical equipment, and pharmaceutical industry. For example, a surgeon can monitor a volume via images (similar to virtual reality (VR) images) on a display during a medical treatment such as a surgery. Because the dosage forms provided by the present invention produce no radiation, patients can be injected with the dosage form in the ward during the pre-operative evaluation and then go to the operating room. Alternatively, a physician can perform a lymph node metastases (LNMets) evaluation for a patient in outpatient, and the patient does not stay any nights in the hospital. In addition, the provided fluorescence imaging method and system can also be employed for postoperative check and/or metastasis evaluation.

The dosage form, fluorescence imaging method and system provided by the present invention can also be applied to health examination. The medical examination may include, but is not limited to: sentinel lymphatic imaging and cancer metastasis evaluation of internal organs.

In addition, some embodiments of the present invention provide a medical equipment using the provided dosage form, fluorescence imaging method, and/or fluorescence imaging system. In a particular example, the medical equipment includes a device used in a minimally invasive surgery or a Da Vinci surgical system.

Furthermore, the provided dosage form, the fluorescence imaging method, and the fluorescence imaging system protect the hospital from radioactive contamination and protect patients from radiation exposure. In terms of diagnostic efficiency, the two-photon absorption dosage form improves the image resolution. When combined with a robot-assisted surgery or a smart surgery, it can greatly reduce injury produced by the surgery and shorten the recovery time. It is a win-win situation for doctors, hospitals, and patients.

Although specific embodiments have been illustrated and described, it will be appreciated by those skilled in the art that various modifications may be made without departing from the scope of the present invention, which is intended to be limited solely by the appended claims.

What is claimed is:

1. A fluorescence imaging method, comprising steps of:
treating an animal or a human body with a dosage form comprising a dye and a polymer for encapsulating the dye;
irradiating a volume of the animal or the human body with an excitation light, wherein an emission light with a peak wavelength equal to or greater than 800 nm is generated by a two or multi-photon absorption of the dosage form, and
detecting a fluorescence emission light from the volume to obtain a fluorescence image including the volume.

2. The fluorescence imaging method as recited in claim 1, further comprising:
irradiating the volume of the animal or the human body by a visible light and detecting a light from the volume to obtain a bright field image; and
merging the fluorescence image and the bright field image to obtain a merged image.

3. The fluorescence imaging method as recited in claim 1, wherein the polymer comprises a functional group for binding to a specific cell in the animal or the human body.

4. The fluorescence imaging method as recited in claim 1, further comprising doping at least one chemical during the treatment for the animal the human body with the dosage form.

5. The fluorescence imaging method as recited in claim 1, wherein the excitation light comprises a continuous near infrared light or a pulsed near infrared light.

6. The fluorescence imaging method as recited in claim 1, wherein the dye comprises quinoxaline derivatives, 2,1,3-benzothiadiazole (BTD) derivatives, or a combination thereof.

7. The fluorescence imaging method as recited in claim 1, wherein the polymer is derived from one or more of the following: poly(ethylenimine), poly(aspartic acid), poly(acrylic acid), dextran, cyclodextran, cyclodextrin, poly(ethylene glycol) (PEG), poly(ethylene oxide) (PEO), poly(oxyethylene) (POE), poly(propylene oxide) (PPO), methoxy poly(ethylene glycol) (mPEG), poly ε-caprolactone (PCL), methoxy poly(ethylene glycol)-poly ε-caprolactone (mPEG-PCL), poly(dithienyl-diketopyrrolopyrrole) (PDPP), poly (N-vinyl pyrrolidone) (PVP), poly (N-isopropyl acrylamide) (pNIPAAm), poly(propylene oxide) (PPO), poly (L-lactide), poly(lactide-co-glycolic acid) (PLGA), poly(L-aspartic acid) (pAsp), poly(L-histidine), poly (β-amino ester) (PbAE), hydrophobic phospholipid residues, disteroyl phosphatidyl ethanolamine (DSPE), poly(ethylene glycol)-b-poly(lactide) (PELA), poly(ethylene glycol)-b-poly(D,L-lactide)-b-poly(β-amino ester) (PELA-PBAE), N-decanoyl-N-methylglucamine (MEGA-10), Pluronic® F-127 (CAS: 9003-11-6), sodium dodecyl sulfate (SDS), sodium dodecylbenzenesulfonate (SDBS), alkyltrimethylammoniun bromides C₁₀TAB, C₁₂TAB, C₁₄TAB, C₁₆TAB, alkyltriphenylphosphonium bromides (C₁₂TPB, C₁₄TPB and C₁₆TPB), or a combination thereof.

8. A fluorescence imaging method, comprising steps of:
treating an animal or a human body with a dosage form comprising a dye and a polymer for encapsulating the dye;
irradiating a volume of the animal or the human body with an excitation light, wherein an emission light with a maximum two-photon cross section larger than 1500 GM within a wavelength range from 700 nm to 1000 nm is generated by a two or multi-photon absorption of the dosage form, and
detecting a fluorescence emission light from the volume to obtain a fluorescence image including the volume.

9. A fluorescence imaging system, comprising:
a dosage form comprising a dye and a polymer for encapsulating the dye and applicable to a treatment of an animal or a human body;
a first light source for irradiating an excitation light on a volume of the animal or the human body, wherein an emission light with a peak wavelength equal to or greater than 800 nm, or an emission light with a maximum two-photon cross section larger than 1500 GM within a wavelength range from 700 nm to 1000 nm is generated by a two or multi-photon absorption of the dosage form; and
a fluorescence image detecting unit for detecting a fluorescence emission light from the volume to obtain a fluorescence image containing the volume.

10. The fluorescence imaging system as recited in claim 9, further comprising:
a second light source for irradiating a visible light on the volume of the animal or the human body; and
a bright-field image detecting unit for detecting a light from the volume to obtain a bright field image including the volume.

11. The fluorescence imaging system as recited in claim 10, further comprising an image processing system for merging the fluorescence image and the bright field image into a merged image.

12. A fluorescence imaging method, comprising steps of:
treating an animal or a human body with a dosage form comprising a dye and a polymer for encapsulating the dye;
irradiating a volume of the animal or the human body by an excitation light, wherein an emission light with a peak wavelength equal to or greater than 780 nm is generated by a single photon absorption of the dosage form; and
detecting a fluorescence emission light from the volume to obtain a fluorescence image including the volume.
